(19) United States
(12) Patent Application Publication
(10) Pub. No.: US 2021/0164411 A1
(43) Pub. Date: Jun. 3, 2021

(54) Method for Determining a Parameter Characterizing the Anti-Knock Property of a Fuel and Corresponding Test System

(71) Applicant: ROFA LABORATORY & PROCESS ANALYZERS, KRITZENDORF (AT)

(72) Inventors: Karl Huber, Eichstaett (DE); Johann Hauber, Neuburg (DE)

(21) Appl. No.: 17/092,641

(22) Filed: Nov. 9, 2020

Related U.S. Application Data
(63) Continuation of application No. 15/144,965, filed on May 3, 2016, now abandoned.

(30) Foreign Application Priority Data
May 4, 2015 (DE) 10 2015 106 881.8

(51) Int. Cl. F02D 41/14; G01N 33/28; F02B 77/08; F02D 35/02

(52) U.S. Cl. CPC F02D 41/1405; G01N 33/2829; F02B 77/085; F02D 2041/1437; F02D 41/1439; F02D 41/1498; F02D 2041/1432; F02D 35/027

(57) ABSTRACT

The disclosure relates to a method for determining a parameter characterizing the anti-knock property of a fuel using a test engine having at least one cylinder, wherein the fuel undergoes combustion inside the cylinder during the course of the method and the cylinder pressure generated by the combustion is detected using a pressure sensor. The pressure sensor has a linear pressure output signal curve. A parameter characterizing the anti-knock property of the fuel is calculated based on the output signal of the pressure sensor. The calculation is done using a mathematical model that considers the deviation of the pressure output signal curve of the pressure sensor being used from the pressure output signal curve of a pick-up sensor prescribed in the ASTM D2699 standard. Moreover, a test system to determine the anti-knock property of a fuel is disclosed.

### CROSS-REFERENCE TO RELATED APPLICATIONS

This application is a continuation of and claims benefit to U.S. patent application Ser. No. 15/144,965, filed May 3, 2016, which claims benefit to German Patent Application No. 10 2015 106 881.8, filed May 4, 2015, both of which are incorporated by reference herein.

### TECHNICAL FIELD

The present disclosure relates to a method for determining a parameter characterizing the anti-knock property of a fuel using a test engine having at least one cylinder, wherein the fuel undergoes combustion inside the cylinder during the course of the method and the cylinder pressure generated by the combustion is detected using a pressure sensor, and wherein a pressure sensor with a linear pressure output signal curve is used.

Furthermore, a test system to determine a parameter characterizing the anti-knock property of a fuel with a test engine having at least one cylinder and a pressure sensor having a linear pressure output signal curve is suggested, wherein the cylinder pressure that prevails while the fuel undergoes combustion in the cylinder is detected using the pressure sensor.

### BACKGROUND

The anti-knock property is one of the most important quality features of gasoline fuels and standardized test procedures are applied to determine it in engines. These procedures were developed more than 80 years ago and have remained almost unchanged in the world's fuel testing laboratories.

In the automotive field, the finite nature of fossil fuels is promoting an ever growing diversification of the types of drives and energy carriers. Especially in liquid hydrocarbons, it must be assumed that the worldwide supply of various fuels with biogenic mixtures will keep increasing. This also applies to the increasing importance of fuel testing because fuel quality must be regarded as a prerequisite for efficient and low-emission combustion.

In gasoline engines, the anti-knock property of the fuel is an extremely important quality feature, as it directly influences the achievable degree of effectiveness through possible compression occurring during already the construction design and through the ignition angle intervention of knocking control while the engine is running. The necessity to test gasoline fuels for their anti-knock property was recognized as early as in the 1930s due to engine damage, and a test procedure was established on the market to determine the anti-knock property.

The parameters that characterize the anti-knock property (octane numbers) are determined according to the ASTM D2699 (research octane number: RON) and D2700 (motor octane number MON) standards and their European equivalents EN-ISO 5163 and EN-ISO 5164 on a standardized test engine by so-called calibration by bracketing with isooctane and n-heptane mixtures. All such standards are incorporated by reference herein.

Generally, in this context, a cooperative fuel research motor (CFR motor) developed by Waukesha is used, which is a one-cylinder test engine having two-valve technology and lateral spark plug position. It is above all characterized by the compression ratio, which can be changed while it runs. The motor has external mixture formation by means of the carburetor and air pre-heating and, in addition, a mixture heater in the MON procedure. According to the standard, the octane number of a fuel is determined through calibration by bracketing with reference fuels. To achieve this, the proper functioning of the test engine must be tested using toluene standardization fuels (TSF), with which the suction temperature must be adjusted, among other things.

To determine the octane number of an unknown fuel, the operator must adjust the air ratio in such a way that the knocking taking place during combustion is at a maximum level. To calibrate by bracketing, two fuel mixtures with known octane number are needed. For octane numbers below 100, two-component mixtures from the more anti-knock and eponymous isooctane (2,2,4-trimethylpentane) and the more easy-knocking n-heptane are used. From these two mixtures, a higher deflection in the knock meter (a moving coil instrument, cf. also FIG. 2 and the description below) with one mixture, and a lower deflection with the other one must be achieved in order to linearly interpolate the octane number of the fuel sample lying in between.

However, one of the problems here is the determination of RON values over 100 because to do this, leaded reference fuels (isooctane with additions of lead tetraethyl) are provided, but they pose significant health risks for the operating staff and therefore they are only limited to small quantities in Europe. For this reason and contrary to the standard, calibration by bracketing takes place in many laboratories with TSF fuels, which should actually be used only for finding out the calibration point.

Since the knock meter indication is the most important value when determining RON and MON, the following paragraphs will deal more deeply with signal detection with the so-called pickup sensor, with signal analysis in the so-called detonation meter, and with the indication of knocking intensity in the knock meter, as provided in the above-mentioned standards.

The pickup sensor associated with the cylinder chamber of the test engine that is used consists essentially of coil-wrapped, magnetostrictive rod. When the rod is deformed as a result of cylinder pressure, a voltage is induced in the rod. This is the only measurable value included in the evaluation logic of the detonation meter, which is an analog circuit whose task is to process the pickup signal and generate a voltage from it whose magnitude is a measure of knocking intensity and is indicated by the knock meter.

Research has now shown that the output signal emitted by the pickup sensor does not behave proportionally to the actual cylinder pressure over the entire relevant measurement range, so that the values displayed by the knock meter do not always indicate a correct measure for the anti-knocking property of the analyzed fuel.

An alternative method for determining a parameter that characterizes the anti-knock property is therefore described in WO 2009/130254 A1, in which the cylinder pressure is measured with a pressure sensor whose output signal behaves proportionately to the cylinder pressure (the pressure sensor has therefore a linear pressure output signal curve). Finally, the determined pressure signals are evaluated statistically to generate in the end a parameter that allows a reliable evaluation of the actual anti-knock property of the respective fuel.

However, the disadvantage is that the parameters determined according to WO 2009/130254 A1 no longer coincide with the parameters determined according to the above-mentioned standards.

The task of the present invention is to address this disadvantage.

### SUMMARY

The task is solved by a method and a test system having the characteristics of the present disclosure.

According to the disclosure, the method is characterized by the fact that a parameter characterizing the anti-knock property of the fuel analyzed in the test engine that is used is calculated based on the output signal of the pressure sensor. In this case, the calculation is done based on a mathematical model that takes into account the deviation of the pressure output signal curve of the pressure sensor being used from the pressure output signal curve of a pickup sensor prescribed in the ASTM D2699 and D2700 standards (wherein within the scope of the present invention, the mentioned standard is the version valid on the priority date of this invention; ASTM stands for the American Society for Testing and Materials).

In other words, the output signal of the pressure sensor (which behaves differently from the signal generated by the above-mentioned pickup sensor in proportion to the actual pressure prevailing inside the cylinder) is used as an input value of a mathematical model for finally calculating a parameter characterizing the anti-knock property of the fuel.

Ultimately, the parameter has the same magnitude that it would have had if the test engine would have been equipped with the above-mentioned pickup sensor and the signal delivered by the pickup sensor would have been processed further according to one of the standards mentioned above. In the final analysis, a parameter (especially in the form of an octane number) is obtained with the method according to the invention that reflects the anti-knock property of the analyzed fuel, whereas contrary to the above-mentioned standards, a pressure sensor is used whose output signal behaves proportionately to the existing cylinder pressure.

Thus, it is finally possible using the test system described in WO 2009/130254 A1 to determine the anti-knock property of a fuel, wherein the anti-knock property determined coincides with the anti-knock property that would have been obtained if one of the above-mentioned standards would have been complied with (whether the anti-knock property is now the RON or MON depends essentially on setting the various test engine parameters such as its rotational speed and therefore has no influence on the applicability of the present invention).

Preferably, the RON or MON is determined using the method according to the invention or the test system according to the invention, wherein the mathematical model is chosen in such a way that the value of the corresponding parameters equals the value that would have been obtained if one of the preceding standards would have been complied with.

It is very advantageous for the mathematical model to include a transfer function with which the parameter characterizing the anti-knock property can be calculated from the output signal of the pressure sensor. Thus, the transfer function serves to convert the output signal of the pressure sensor into the corresponding parameter (RON or MON), wherein the transfer function is procured in such a way that the parameter obtained corresponds in its amount to the parameter that would have been determined if one of the above-mentioned standards would have been complied with (i.e., when using a pickup sensor).

It is advantageous if the transfer function is developed as differential equation or includes such equation. In particular, it is useful here to supply certain signal portions of the output signals of the pressure sensor to the differential equation as input value. For example, it is conceivable that only the signal or signal portions are used that are provided by the pressure sensor during maximum cylinder pressure.

It is additionally advantageous if the differential equation includes time derivatives of different orders of the output signal of the pressure sensor or the cylinder pressure determined from this. In particular, the first derivative (i.e., the pressure change over time) delivers information that allows conclusions to be drawn about the anti-knock property of the analyzed fuel.

It is likewise also advantageous if the time derivatives are weighed differently within the differential equation. For example, it could be conceivable to multiply all or individual derivatives by a factor, wherein the respective factors have, at least partially, different values. Depending on the amount of the value of the cylinder pressure that was determined (i.e., depending on the amount of the value of the output signal of the pressure sensor), the output signal is corrected to a varying extent so that ultimately a parameter is determined whose amount does not differ from the parameter that would have been obtained if the same fuel sample would have been analyzed according to one of the previous standards.

It is also extremely advantageous if the pickup sensor prescribed in the ASTM D2699 or D2700 standard is simulated using the mathematical model by correcting the output signal of the pressure sensor in such a way that the corrected signal corresponds substantially to the output signal of the pickup sensor prescribed in the ASTM D2699 or D2700 standard with the same cylinder pressure. Finally, the corrected output signal could be supplied to a detonation meter described in the above-mentioned standards, which eventually forwards a signal dependent on the output signal to the knock meter that is also mentioned in the above-mentioned standards. Finally, the knock meter indicates the desired knock intensity that provides, by means of linear interpolation, the octane number to the calibration by bracketing that would have been obtained if one of the above-mentioned standards would have been fully complied with (i.e. when a pickup sensor is used instead of the pressure sensor with linear pressure output curve according to the invention).

It is likewise advantageous if the measuring chain consisting of pickup sensor, detonation meter and knock meter is simulated using the mathematical model described in the ASTM D2699 and D2700 standards. Thus, the mathematical model forms the above-mentioned measuring chain again, wherein the cylinder pressure determined by the pressure sensor serves as input value for the simulated measuring chain to ultimately calculate the corresponding octane number (RON or MON).

Special advantages result when the output signal of the pressure sensor is filtered using a low-pass filter to remove the frequencies of the output signal whose value has exceeded a certain limit, wherein the parameter characterizing the anti-knock property of the fuel is calculated based on the filtered output signal.

It is especially advantageous if a piezoelectric pressure sensor is used as pressure sensor because such sensors stand out owing to their own very high frequency and excellent linearity.

The use of an artificial neuronal network is advantageous to evaluate the output signal of the pressure sensor. Using the neuronal network, it is possible, based on a corresponding learning dataset (relationship between the output signal of the pressure sensor and the respective octane number of the fuel sample to be analyzed taking one of the above-mentioned standards into account, wherein many different types of fuel are analyzed), to develop a model used as basis to finally convert the output signal of the pressure sensor (which is detected during the combustion of the fuel sample to be analyzed) to the corresponding octane number (RON or MON).

Finally, a test system is disclosed to execute the described method.

The test system serves to determine one parameter that characterizes the anti-knock property of a fuel with a test engine, where the test engine has at least one cylinder and one pressure sensor with a linear pressure output signal curve, and wherein the pressure sensor allows the detection of the cylinder pressure that prevails in the cylinder during the combustion of the fuel.

According to the disclosure, it is suggested for the test system to comprise an evaluation unit with whose help (or the one of a software installed in the evaluation unit) a parameter characterizing the anti-knock property of the fuel can be calculated based on the output signal of the pressure sensor. The calculation is done based on a mathematical model that considers the deviation of the pressure output signal curve of the pressure sensor used from the pressure output signal curve of a pickup sensor described in the ASTM D2699 or D2700 standard.

Generally, in this case, the evaluation unit can be designed to execute the process described so far, wherein any combination of individual characteristics can become reality (as long as this does not lead to incompatibilities).

In particular, the test system can therefore also have a low-pass filter or a pressure sensor designed as piezo pressure sensor. Thus, it is not necessary to use the pickup sensor defined in the ASTM D2699 or D2700 standard.

### BRIEF DESCRIPTION OF THE DRAWINGS

Additional specific aspects of the invention are described and shown below:

FIG. 1 is a schematic view of a test engine used to determine the anti-knock property of a fuel as used, for example, according to the ASTM D2699 (EN ISO 5163) or D2700 (EN ISO 5164) standard;

FIG. 2 is a schematic view of the measuring chain described in the ASTM D2699 (EN ISO 5163) or D2700 (EN ISO 5164) standard; and

FIG. 3 is a schematic view of a test system according to the disclosure used to execute the method according to the disclosure.

### DETAILED DESCRIPTION

Typically, the determination of octane numbers is carried out empirically throughout the world according to standardized procedures in the laboratories of fuel producers, where special one-cylinder test engines with a variable compression ratio are used to set the corresponding fuel quality.

The objective is to compare the knock intensity of the fuel to be tested with that of fuels with known octane numbers and determine the octane numbers through interpolation, if applicable. In the standard, isooctane was arbitrarily allocated the octane number of 100 and n-heptane the octane number of 0. By mixing these components, it is possible to manufacture a fuel having the same knock intensity as the fuel to be tested. Then, the octane number being sought corresponds to the volumetric proportion of the isooctane in the fuel mixture. Testing conditions differentiate between MON and RON, although all other procedural steps coincide and even the same measuring technology and the same test engine 2 are used.

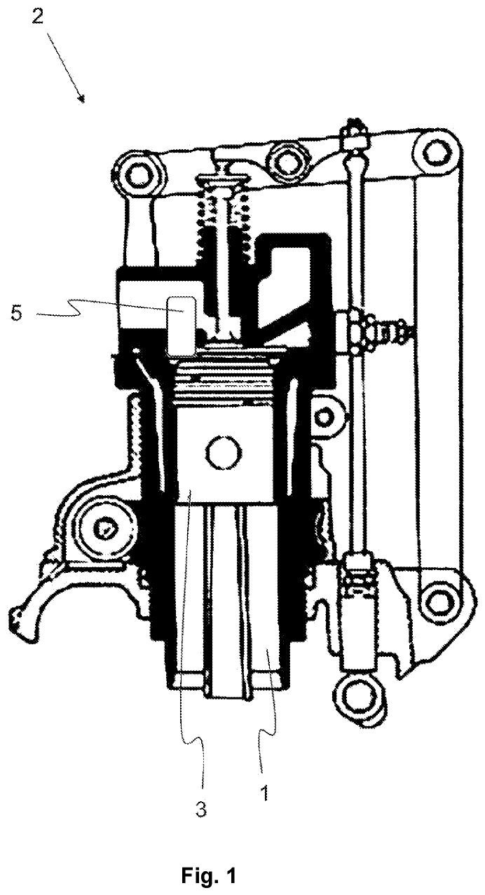

FIG. 1 shows a test engine 2 to determine the anti-knock property of a fuel used according to the ASTM D2699 or D2700 standard (determination of the RON or MON), for example.

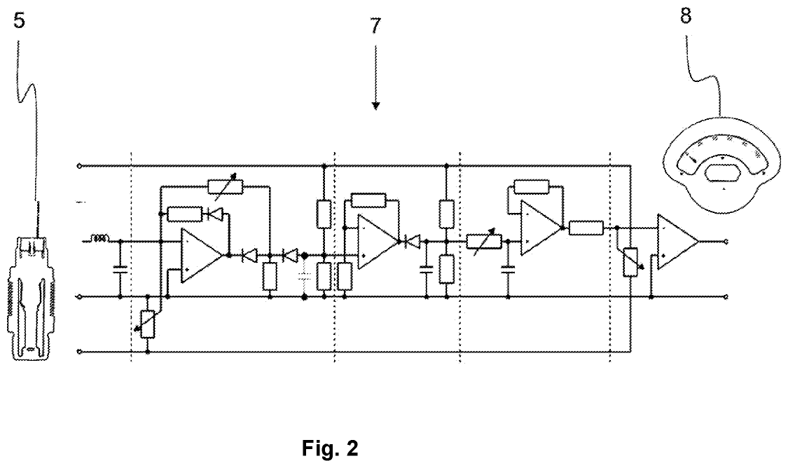

The extent of knock intensity is generated through an electric sensor (pickup sensor 5) screwed firmly into the engine's combustion chamber (FIG. 1) and displayed via an analog circuit, the so-called detonation meter 7, on an indicator instrument (knock meter 8) (FIG. 2).

Applicant's own research has revealed that the signal provided by the pickup sensor 5 is not proportional to the cylinder pressure, so that an accuracy of no more than +/−0.2 octane numbers can be achieved with the above-mentioned procedure.

Furthermore, WO 2009/130254 A1 describes a method in which the cylinder pressure is measured with a pressure sensor 4 whose output signal behaves proportionately to the cylinder pressure (hence, the pressure sensor 4 has a linear pressure output signal characteristic curve). The pressure signals determined are statistically evaluated to finally generate a parameter that allows a reliable evaluation of the actual anti-knock property of the respective fuel.

However, the disadvantage of the method described there is that the parameters that are determined do not coincide with the octane numbers obtained when the above-mentioned standards are observed.

So an octane number that coincides with the octane number that would have been determined if one of the above-mentioned standards had been observed can also be determined now with the test system described in WO 2009/130254 A1, the suggestion is now made—based on the output signal of the pressure sensor 4 used (which has a linear pressure output signal curve)—to calculate a parameter characterizing the anti-knock property of the fuel, wherein the calculation takes place based on a mathematical model that takes into account the deviation of the pressure output signal curve of the pressure sensor 4 that is used from the pressure output signal curve of a pickup sensor 5 prescribed in the ASTM D2699 or D2700 standard.

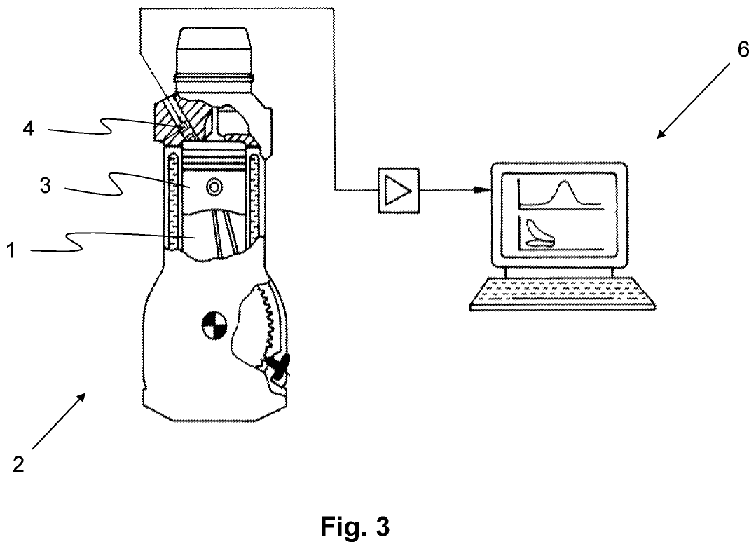

Therefore, according to the disclosure, a test engine 2 is used (as shown schematically in FIG. 3), wherein the test engine 2 has a cylinder 1, a piston 3 within the cylinder 1, and a pressure sensor 4 to detect the cylinder pressure generated during fuel combustion (wherein the pressure sensor 4 is designed so that its output signal behaves proportionally to the cylinder pressure).

Moreover, the pressure sensor 4 is connected to an evaluation unit 6 (e.g., a personal computer) with installed evaluation software to evaluate the output signal according to the invention's method described above and convert it to an octane number.

The general essence of the disclosure is therefore to determine an octane number with a test system not using a pickup sensor 5 prescribed in the ASTM D2699 (EN ISO 5163) or D2700 (EN ISO 5164) standard, which number corresponds to the octane number that would have been obtained if the respective fuel sample would have been analyzed according to one of the above-mentioned standards and thus using a pickup sensor 5.

### LIST OF REFERENCE CHARACTERS

1 Cylinder
2 Test engine
3 Piston
4 Pressure sensor
5 Pickup sensor
6 Evaluation unit
7 Detonation meter
8 Knock meter

1. A method for determining a parameter characterizing an anti-knock property of a fuel using a test engine having at least one cylinder, the method comprising the steps of:
combusting the fuel inside the cylinder;
detecting a cylinder pressure generated by the combustion using a pressure sensor with a pressure output signal curve that is linear;
calculating the parameter based on an output signal of the pressure sensor based on a mathematical model that takes into account a deviation of the pressure output signal curve of the pressure sensor from a pressure output signal curve of a pickup sensor prescribed in the ASTM D2699 standard;
wherein the mathematical model comprises a transfer function with which the parameter characterizing the anti-knock property is calculated from the output signal of the pressure sensor, the transfer function includes use of a differential equation, the differential equation includes one of time derivatives of different orders of the output signal of the pressure sensor or the cylinder pressure determined from the time derivatives, and the time derivatives are weighted differently within the differential equation; and
simulating the pickup sensor prescribed in the ASTM D2699 standard using the mathematical model by correcting the output signal of the pressure sensor in such a way that a corrected signal corresponds fundamentally to the output signal of the pickup sensor prescribed in the ASTM D2699 standard under an identical cylinder pressure.

2. A test system to determine a parameter characterizing the anti-knock property of a fuel, the test system comprising:
a test engine having at least one cylinder and a pressure sensor with a pressure output signal curve that is linear, wherein the cylinder pressure that prevails during the combustion of the fuel in the cylinder is detected using the pressure sensor;
an evaluation unit with which the anti-knock property of the parameter characterizing the anti-knock property of the fuel can be calculated, based on an output signal of the pressure sensor, and based on a mathematical model that takes into account the deviation of the pressure output signal curve of the pressure sensor from a pressure output signal curve of a pickup sensor prescribed in ASTM D2699 standard;
wherein the mathematical model comprises a transfer function with which the parameter characterizing the anti-knock property is calculated from the output signal of the pressure sensor, the transfer function includes use of a differential equation, the differential equation includes one of time derivatives of different orders of the output signal of the pressure sensor or the cylinder pressure determined from the time derivatives, and the time derivatives are weighted differently within the differential equation; and
simulating the pickup sensor prescribed in the ASTM D2699 standard using the mathematical model by correcting the output signal of the pressure sensor in such a way that a corrected signal corresponds fundamentally to the output signal of the pickup sensor prescribed in the ASTM D2699 standard under an identical cylinder pressure.

3. The method according to claim 1, further comprising simulating a measuring chain consisting of the pickup sensor, a detonation meter and a knock meter described in the ASTM D2699 standard is simulated using the mathematical model.

4. The method according to claim 1, further comprising filtering the output signal of the pressure sensor using a low-pass filter to create a filtered output signal, and that the parameter characterizing the anti-knock property of the fuel is calculated based on the filtered output signal.

5. The method according to claim 1, wherein the pressure sensor is a piezoelectric pressure sensor.

6. The method according to claim 4, wherein the pressure sensor is a piezoelectric pressure sensor.

7. The method according to claim 1, wherein an evaluation of the output signal of the pressure sensor is done using an artificial neuronal network, wherein the artificial neuronal network uses a relationship between the output signal of the pressure sensor and respective octane number of the fuel to be analyzed to analyze a plurality of different types of fuels, wherein the octane number of the fuel to be analyzed is obtained by considering the ASTM D2699 standard or the D2700 standard.

8. The method according to claim 4, wherein an evaluation of the output signal of the pressure sensor is done using an artificial neuronal network, wherein the artificial neuronal network uses a relationship between the output signal of the pressure sensor and respective octane number of the fuel to be analyzed to analyze a plurality of different types of fuels, wherein the octane number of the fuel to be analyzed is obtained by considering the ASTM D2699 standard or the D2700 standard.

9. The method according to claim 5, wherein an evaluation of the output signal of the pressure sensor is done using an artificial neuronal network, wherein the artificial neuronal network uses a relationship between the output signal of the pressure sensor and respective octane number of the fuel to be analyzed to analyze a plurality of different types of fuels, wherein the octane number of the fuel to be analyzed is obtained by considering the ASTM D2699 standard or the D2700 standard.
